(19) United States
(12) Patent Application Publication
(10) Pub. No.: US 2006/0137107 A1
(43) Pub. Date: Jun. 29, 2006

(54) Operating method of laundry device

(75) Inventors: Deug Hee Lee, Gimhae-si (KR); Hung Myong Cho, Jillye-myeon (KR); Byung Hwan Ahn, Gimhae-si (KR); Soung Bong Choi, Changwon-si (KR); Young Soo Kim, Changwon-si (KR)

Correspondence Address: MCKENNA LONG & ALDRIDGE LLP, 1900 K STREET, NW, WASHINGTON, DC, 20006, US

(73) Assignee: LG ELECTRONICS, INC., Youngdungpo-gu (KR)

(21) Appl. No.: 11/289,653

(22) Filed: Nov. 30, 2005

(30) Foreign Application Priority Data
Nov. 30, 2004 (KR) 10-2004-99244

(51) Int. Cl. A01H 5/02; D06B 19/00; D06F 35/00

(52) U.S. Cl. 8/149.3; 8/148; 8/158

(57) ABSTRACT

An operating method of a laundry device is disclosed. The present invention relates more particularly, to an operation method for a combination dryer which enables air in a drying drum and a cabinet for drying the laundry to circulate continuously and enables water generated in the circulation process to perform a refreshing cycle by using the water.

This application claims the benefit of the Patent Korean Application No. P2004-99244, filed on Nov. 30, 2004, which is hereby incorporated by reference as if fully set forth herein.

### BACKGROUND OF THE INVENTION

1. Field of the Invention

The present invention relates to a laundry device, and more particularly, to a new type of laundry device provided with a washing machine and a cabinet dryer as one body.

2. Discussion of the Related Art

In general, laundry devices including washing machines and dryers are electric home appliances which can wash or dry cloth items, cloths and beddings (hereinafter, “the laundry”).

A dryer of the laundry devices used in a house usually dries a small amount of the laundry.

That is, the dryer receives the laundry washed by the washing machine, and dries the laundry by supplying hot air.

However, the related art dryer has a problem that drying for a tangled portion of the laundry is not dried smoothly, because the drying cycle is in process in a state of the laundry being introduced together at one time.

There is another problem that it is impossible to keep the laundry for a long time in the related art dryer.

Thus, recently demands have been increasing accordingly for a new type of a dryer having a drying capacity thereof enlarged as well as capable of keeping the laundry for a long time. There are various kinds of combination dryers provided with tumble dryers and auxiliary cabinet dryers therewith, for example, U.S. Laid-open Patent Application No. 2004-0194339 A1 or U.S. Laid-open Patent Application No. 2004-0154194.

The above combination dryer allows a cabinet dryer provided on a top of a conventional dryer having a rotatory drum.

The cabinet dryer has space for the laundry and receives hot air used to dry or keep the laundry for a long time.

However, the combination dryer described above has an inconvenience that it performs only a drying cycle and keeping of the laundry, not a washing cycle.

That is, since an auxiliary washing machine should be provided for washing the laundry, a user should purchase a washing machine and a combination dryer separately. Thus, it is difficult to secure an installation space, and production cost may rise due to overlapping of some configurations.

Especially, when it comes to a washing machine with a drying function out of various kinds of washing machines, there is an inconvenience that the laundry should be taken out of right after completing a drying cycle, because the washing machine with a drying function performs only a drying cycle for the laundry.

That is, in spite of completing a drying cycle, when the laundry is kept for a long time there may be too severe wrinkles to iron out easily.

### SUMMARY OF THE INVENTION

Accordingly, the present invention is directed to and an operating method of a laundry device that substantially obviates one or more problems due to limitations and disadvantages of the related art.

An object of the present invention is to provide a new operating method of a laundry device provided with a washing machine with a drying function and a cabinet dryer sharing a structure supplying hot air and steam for washing, drying and refreshing the laundry using the washing machine with a drying function and the cabinet.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, an operating method of a laundry device comprising a first step of identifying a kind of an operation selected by a user out of a washing, a drying and a refreshing cycle; a second step of generating steam by operating a heating element in case the identified operation is a refreshing cycle; and a third step of selectively supplying the steam into a washing machine with a drying function and/or a cabinet dryer using the same drying heater, fan and steam generating part together.

The second step includes a step of identifying an operation object performing a refreshing cycle, and a step of opening a first steam supplying pipe, or a second steam supplying pipe connecting the identified operation object with the steam generating part.

In another aspect of the present invention, an operating method of a laundry device comprising a first step of a washing cycle for washing the laundry by using a washing machine with a drying finction, out of the washing machine with a drying finction and the cabinet dryer using the same drying heater, fan and steam generating part together; a second step of a drying cycle for drying the laundry by using the washing machine with a drying function or the cabinet dryer; and a third step of a refreshing cycle for refreshing the laundry by supplying steam into at least one of the washing machine with a drying function and the cabinet dryer, after drying the laundry.

The first step comprises a step of drying another laundry within the cabinet dryer by using the cabinet dryer.

Also, the first step comprises a step of a refreshing cycle for another laundry within the cabinet dryer by using the cabinet dryer.

Furthermore, the second step comprises a step of a washing, a drying or a refreshing cycle by using one of the washing machine with a drying function and the cabinet dryer, which is not performing a drying cycle.

Still further the third step comprises a step of a washing, drying or refreshing cycle by using one of the washing machine with a drying function and the cabinet dryer which is not performing a refreshing cycle.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a diagram schematically illustrating an exterior of a laundry device according to an embodiment of the present invention.

FIG. 2 is a block diagram schematically illustrating the laundry device according to the embodiment of the present.

FIG. 3 is a flow chart schematically illustrating an operating process of a laundry device according to the first embodiment of the present invention.

FIG. 4 is a flow chart schematically illustrating an operating process of a laundry device according to a second embodiment of the present invention.

FIG. 5 is a flow chart schematically illustrating an operating process of a laundry device according to a third embodiment of the present invention.

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

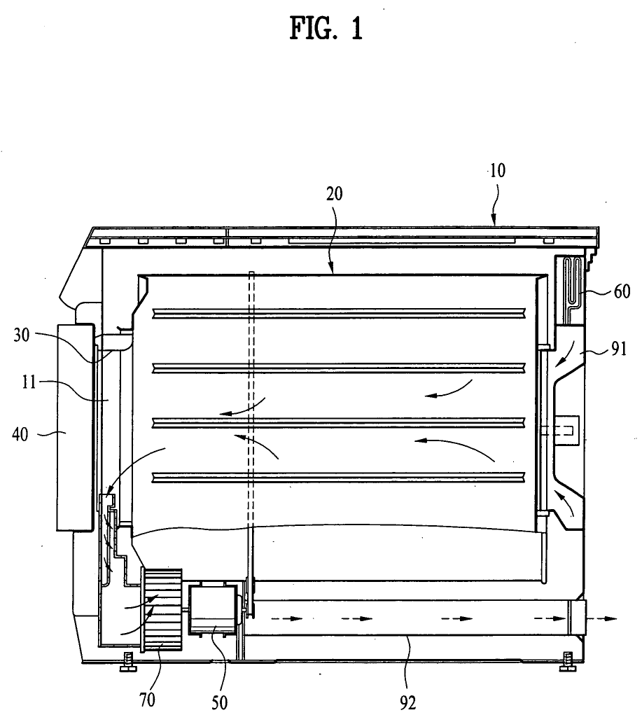

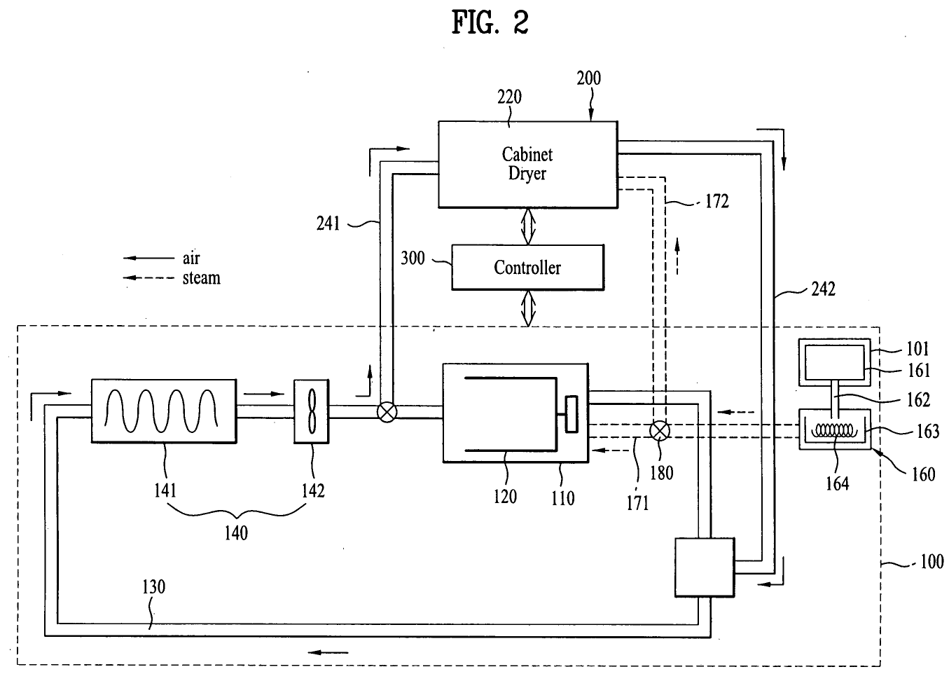

As shown in FIGS. 1 to 2, a laundry device according to an embodiment of the present invention includes a washing machine with a drying function 100, a cabinet dryer 200, and a controller 300.

The washing machine with a drying function 100 will be described.

The washing machine with a drying function 100 includes a tub 110, a drum 120, a hot air supplying pipe 130, hot air supplying part 140 and a steam generating part 160. The washing machine with a drying function performs a washing cycle and a drying cycle.

The drum 120 capable of rotating/agitating is mounted within the tub 110 for performing a washing and a drying cycle in a state of the laundry being introduced.

The hot air supplying pipe 130 as a pipe guiding an inflow of hot air into the drum 120 is formed for re-supplying air into the tub 110 after receiving and circulating the air within the tub 110.

Also, the hot air supplying part 140 is provided within the hot air supplying pipe 130.

The hot air supplying part 140 includes a drying heater 141 for heating flowing air, and a fan 142 for forcibly ventilating the air within the hot air supplying pipe 130.

At that time, the fan 142 may be provided at an air outlet of the hot air supplying pipe 130 where the air of the drying heater 141 is discharged.

That is for preventing damage of a fan motor (not shown) in advance which may be caused when the air containing much moisture within the hot air supplying pipe 130 passes through the fan motor driving fan 142.

Alternatively, the fan 142 may be provided at an air inlet of the hot air supplying pipe 130 where the air of the drying heater is drawn with respect to a direction of an air inflow.

The steam generating part 160 generates steam and supplies the steam into the tub 110 (or the drum 120) of the washing machine with a drying function 100 and the cabinet dryer 200.

The steam generating part 160 and the washing machine with a drying function 100 are connected by a first steam supplying pipe 171, and the steam generating part and the cabinet dryer 200 are connected by a second steam supplying pipe 172.

Furthermore, an air channel valve 180 is provided at a branched portion between the first and second steam supplying pipes 171 and 172 for selectively opening/closing the first and second steam supplying pipes 171 and 172. The air channel valve 180 may be a 3-way valve.

The steam generating part 160 includes a water chamber 161 for storing water, a water supplying pipe 162 connected with the water chamber 161 for receiving the water from the water chamber 161, a heat radiating part 163 for temporarily storing the water received from the water chamber 161, and a heating element 164 provided within the heat radiating part 163 for heating and evaporating the stored water into steam.

Preferably, the steam generating part 160 is provided at one of corners in upper space of the washing machine with a drying function 100. That is why the steam generating part 160 may be provided safely without any interferences of the drum 110.

Especially, a user should replenish the water chamber 161 of the steam generating part 160 with water. Thereby, a water chamber holding part 101 is further provided at an upper corner of the washing machine with a drying function 100 for holding the water chamber 161 therein as well as being replenished with water.

As described before, hot air generated by operating the hot air supplying part 140 and steam generated by operating the steam generating part 160 are used by the cabinet dryer 200 as well as the washing machine with a drying function 100, which will be described. That will be re-described more specifically in the detailed description of the cabinet dryer 200.

Next, the cabinet dryer 200 will be described.

The cabinet dryer 200 includes a keeping part 220 for keeping the laundry therein, and is mounted on a top of the washing machine with a drying function 100.

The cabinet dryer 200 includes a hot air inlet pipe 241 and an air outlet pipe 242.

The hot air inlet pipe 241 receives hot air flowing in the hot air supplying pipe 130, and guides the hot air into the keeping part 220.

A first end of the hot air inlet pipe 241 is connected with the keeping part 220, and a second end thereof is connected with the hot air supplying pipe 130.

Preferably, a hot air inlet of the hot air inlet pipe 241 is connected with an air outlet of the fan 142 in the hot air supplying pipe 130.

Also, the air inlet pipe 242 supplies air containing moisture, after flowing within the keeping 220, into the hot air supplying pipe 130. Then, an air inlet of the air outlet pipe 242 is connected with the keeping part 220, and an air outlet of the air outlet pipe 242 is connected with the hot air supplying pipe 130.

Next, the controller 300 will be described.

The controller 300 controls the washing machine with a drying function 100 and the cabinet dryer 200

At that time, the controller 300 is provided in at least one of the washing machine with a drying function 100 and the cabinet dryer 200, but preferably provided only in the washing machine with a drying function 100 as shown in the embodiment of the present invention.

If the controller 300 is provided in both the washing machine with a drying function 100 and the cabinet dryer 200, the controller 300 are connected by a data cable (not shown) to make it possible to intercommunicate information.

Also, the controller 300 may control the washing machine with a drying function 100 and the cabinet dryer 200 respectively, and may control the washing with a drying function 100 and the cabinet dryer 200 to communicate each other.

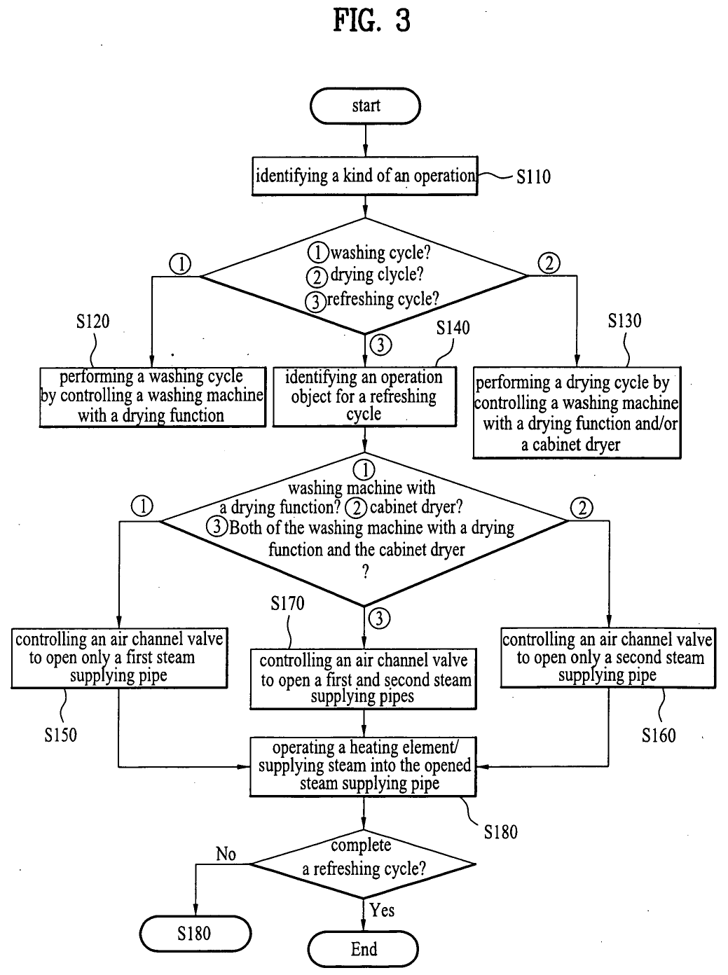

Referring to FIG. 3, an operating method of a laundry device according to a first embodiment of the present invention will be described.

First of all, the controller 300 identifies a kind of cycles the user select (S10).

The kinds of the cycles include a washing, a drying and a refreshing. 9.

Next, based on the identified kind of the cycles, the controller 300 performs the selected cycle by selectively controlling the washing machine with a drying function 100 and/or the cabinet dryer 200.

Each kind of the cycles will be described in detail.

First, a case of a washing cycle will be described.

The washing cycle is performed by the controller 300's control of the washing machine with a drying function 100.

That is, the washing machine with a drying function 100 is controlled for washing the laundry (S120).

The control for washing the laundry is operated by combination of controlling a driving of the drum 110 and controlling water supply. The washing cycle includes a washing, a rinsing, and a spinning.

Also, when washing the laundry, the cabinet dryer 200 is not operated.

Next, a case of a drying cycle will be described.

The drying cycle is operated by the controller 300's selective control of the washing machine with a drying function 100 and/or the cabinet dryer 200.

That is, the laundry is dried by using the washing machine with a drying function 100 (S130) or using the cabinet dryer 200, or the laundry is dried by controlling the washing machine with a drying function 100 and the cabinet dryer 200.

At that time, the laundry is dried by hot air. The hot air is generated by operating the hot air supplying part of the washing machine with a drying function 100, and supplied into the washing machine with a drying function 100 and/or the cabinet dryer 200 by operating the fan.

Next, a case of a refreshing cycle will be described.

Once the refreshing cycle is selected, the controller 300 identifies an operation object (S140).

At that time, the controller 300 selectively opens the first and second steam supplying pipes 171 and 172 through the control of the air channel valve 180.

The selective opening of the first and second steam supplying pipes 171 and 172 is a series of processes controlling steam to be supplied only into the washing machine with a drying function 100 and/or the cabinet dryer, of which an operation is performed, based on the data identified in the above process.

For example, when the operation object is identified as the washing machine with a drying function 100, the air channel valve 180 is controlled to open only the first steam supplying pipe 171 connected with the washing machine with a drying function 100 (S150).

Whereas, once the operation object is identified as the cabinet dryer 200, the air channel valve 180 is controlled to open only the second steam supplying pipe 172 connected with the cabinet dryer 200 (S160).

Once the operation object is identified as the washing machine with a drying function 100 and the cabinet dryer 200, the air channel valve 180 is controlled to open both of the first and second steam supplying pipe 171 and 172 (S170).

The opening range of the first steam supplying pipe 171 and/or the second steam supplying pipe (or the quantity of steam) may be as changeable as necessary. For example, when a large amount of the steam is needed, the first steam supplying pipe 171 and/or the second steam supplying pipe 172 is/are opened as much as possible. When a small amount of the steam is needed, the first steam supplying pipe 171 and/or the second steam supplying pipe 172 is/are opened relatively little.

As described above, once the first and second steam supplying pipe 171 and 172 are selectively opened based on the identified operation object, the controller 300 radiates heat of the heating element 164 within the heat radiating part 163 of the steam generating part 160. At that time, water is stored in the heat radiating part 163. The water is supplied from the water chamber 161 into the heat radiating part 163 by the water supplying pipe 162.

The water stored within the heat radiating part 163 by the heating element 164 is evaporated, thereby hot steam being generated. The steam is flown through the opened first and/or second steam supplying pipe 171 and 172, and supplied into the washing machine with a drying function 100 and/or the cabinet dryer 200 (S180).

For example, in case only the first steam supplying pipe 171 is opened, the hot steam is supplied only into the washing with a drying function 100 through the first steam supplying pipe 171. In case only the second steam supplying pipe 172 is opened, the hot steam is supplied only into the cabinet dryer 200 through the second steam supplying pipe 172. In case the first and second steam supplying pipes 171 and 172 are all opened, the hot steam is supplied into both the washing machine with a drying function 100 and the cabinet dryer 200 through the first and second steam supplying pipes 171 and 172.

Thus, the laundry within the washing machine with a drying function 100 and/or the cabinet dryer 200 may be refreshed, in other words, lets its wrinkles smoothed out and get sterilized.

When a refresh cycle is selected during the drying cycle of the washing machine with a drying function 100, the controller 300 closes the hot air inlet pipe 241 which supplies hot air into the cabinet dryer 200, and at the same time controls the air. channel valve 180 to open the second steam supplying pipe 172 and radiates heat of the heating element 164 of the steam generating part 160.

Thus, the hot air is supplied into the washing machine with a drying function 100 to perform a drying cycle, and high temperature steam is supplied into the cabinet dryer to perform a refreshing cycle for dried clothes.

On the other hand, an operating method of a laundry device according to a second embodiment of the present invention allows it possible that another cycle using the other one is performed at the same time during one of the washing, drying and refreshing cycles using the washing machine with a drying function 100 or the cabinet dryer 200.

For example, while a washing cycle or a drying cycle using the washing machine with a drying function 100 is getting performed, a refreshing cycle using the cabinet dryer 200 may be performed at the same time.

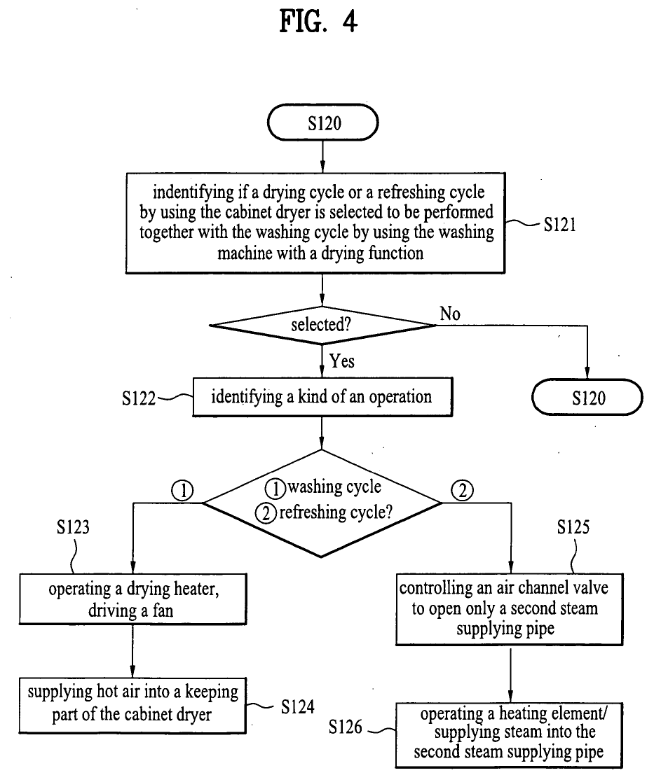

Referring to the flow chart of FIG. 4, the above method will be described more specifically.

First, while a washing cycle using the washing machine with a drying function 100 is performed, the controller 300 consistently identifies a request for controlling the cabinet dryer (S121).

Once the user selects an operation of the cycles during that process, the controller 300 identifies the selected kind of operations (122).

At that time, the kinds of operations includes a drying cycle and a refreshing cycle.

When the selected kind of operation is identified to be a drying cycle using the cabinet dryer 200, the controller 300 operates the drying heater 141 and drives the fan 142.

Hence, hot air is generated within the hot air supplying pipe, and then the hot air is supplied into the keeping part 220 of the cabinet dryer 200 after passing through the hot air inlet pipe 241 by driving the fan 142 (S124). Thus, the laundry within the laundry keeping space 220 is dried.

When the selected kind of operation is identified to be a refreshing cycle using the cabinet dryer 200, the controller 300 controls the air channel valve 180 to open the second steam supplying pipe 172 (S125). Also, the controller 300 operates the heating element 164 of the heat radiating part 163.

Thus, water stored within the heat radiating part 163 is evaporated, and hot steam is generated. Hence, the hot steam is flowing along the opened second steam supplying pipe 172 to be supplied into the keeping part 220 (S126). Thus, the laundry such as clothes is refreshed by the hot steam.

Alternatively, the operating method of the laundry device according to the second embodiment of the present invention allows it possible to perform a refreshing cycle by using the cabinet dryer 200 during a drying cycle using the washing machine with a drying function 100.

In that case, the controller 300 closes the hot air inlet pipe 241 supplying hot air into the cabinet dryer 200. Also, the controller 300 opens the second steam supplying pipe 172 by controlling the air channel valve 180 and operating the heating element 164 of the steam generating part 160.

Thus, hot air is supplied into the washing machine with a drying function 100 to perform a drying cycle, and at the same time hot steam is supplied into the cabinet dryer 200 to perform a refreshing cycle by the hot steam.

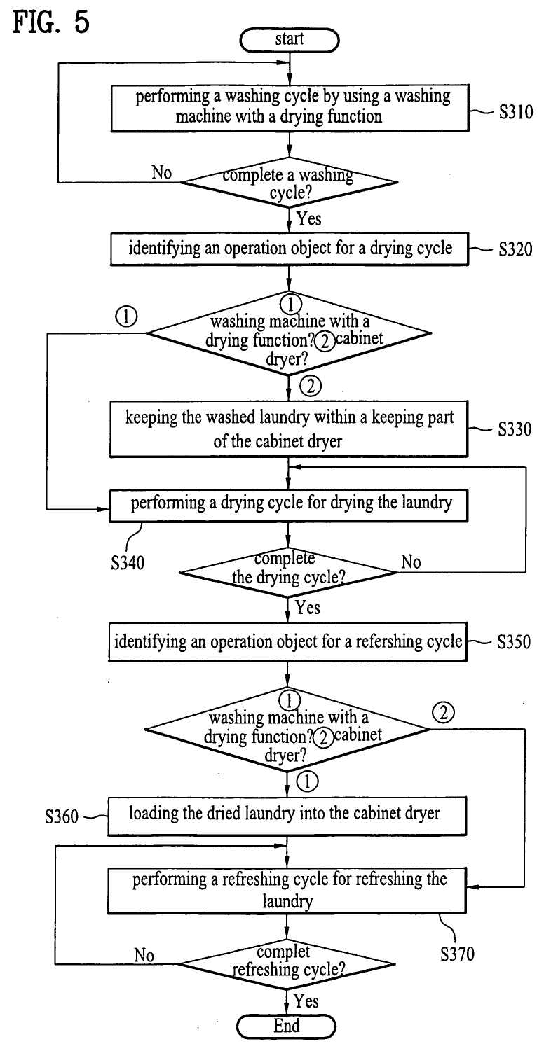

On the other hand, FIG. 5 is a flow chart illustrating an operating method of a laundry device according to a third embodiment of the present invention.

According to the third embodiment of the present invention, the operating method of the laundry device allows a washing, drying and refreshing cycle performed successively.

That operating method classified into 3 processes, as described as follows.

First, a washing cycle is performed by using the washing machine with a drying function 100 (S310).

Second, a drying cycle is performed by using the washing machine with a drying function 100 or the cabinet dryer 200 (S340).

Third, a refreshing cycle is performed by using the washing machine with a drying function 100 or the cabinet dryer 200 (S370).

Therefore, the third embodiment of the present invention has an advantageous effect that washing, drying and refreshing the laundry is performed by one laundry device successively.

The controller 300 identifies an operation object for the next cycle (S320 and S350), and allows the laundry supplied into the identified operation object (S330 and S360).

For example, when a washing cycle is completed, the operation object for a drying cycle is identified to be the cabinet dryer 200. Hence, the laundry of the washing machine with a drying function 100 is moved into the cabinet dryer 200 for performing a drying cycle. That process is the same as a refreshing cycle is performed after completing the drying cycle.

The laundry is moved by the user, and the controller 300 is employed to notify the user of that information.

Alternatively, while performing the washing, drying, or refreshing cycle using the washing machine with a drying function 100 or the cabinet dryer 200, the other one identified not to be the operation object also may performed one of the cycles at the same time.

For example, while performing the washing cycle of the washing machine with a drying function 100, a drying cycle or a refreshing cycle using the cabinet dryer 200 may be performed at the same time.

Furthermore, while performing a drying cycle using the washing machine with a drying function 100, a drying cycle or a refreshing cycle using the cabinet dryer 200 may be performed at the same time. Also, while performing a drying cycle using the washing machine with a drying function 100, a drying cycle or a refreshing cycle using the washing machine with a drying function 100 may be performed at the same time.

Still further, while performing a refreshing cycle using the washing machine with a drying function 100, a drying cycle or a refreshing cycle using the cabinet dryer 200 may be performed at the same time. Alternatively, while performing a refreshing cycle using the cabinet dryer 200, a washing cycle, a drying cycle or a refreshing cycle using the washing machine with a drying function 100 may be performed at the same time.

Especially, of the above series of the processes, it is preferred but not necessary that in the drying cycle the washing machine with a drying function 100 dries the laundry roughly, and then the roughly dried laundry is loaded into the cabinet dryer 200 for re-drying the laundry.

Preferably, the refreshing cycle is performed right after the drying cycle using the cabinet dryer 200.

Alternatively, the refreshing cycle may be performed in the cabinet dryer 200 during the drying cycle using the washing machine with a drying function 100 at the same time, and may be successively performed in the washing machine with a drying function 100 after the drying cycle using the washing machine with a drying function 100.

The operating method of the laundry device according to each embodiment of the present invention may have an advantageous effect that a drying and a refreshing cycle as well as a washing cycle are performed successively.

Especially, even while a washing or a drying cycle using the washing machine with a drying function 100 is performed, a refreshing cycle using the cabinet dryer 200 may be performed at the same time. Thereby, reducing the time taken for the entire washing.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. An operating method of a laundry device comprising:
a first step of identifying a kind of an operation selected by a user out of a washing, a drying and a refreshing cycle;
a second step of generating steam by operating a heating element in case the identified operation is a refreshing cycle; and
a third step of selectively supplying the steam into a washing machine with a drying function and/or a cabinet dryer using the same drying heater, fan and steam generating part together.

2. The operating of the laundry device of claim 1, wherein the laundry is washed by controlling the washing machine with a drying function without performing the second and third steps, once the identified operation is a washing cycle.

3. The operating of the laundry device of claim 1, wherein the laundry is dried by controlling the washing machine with a drying function and/or the cabinet dryer without performing the second and third steps, once the identified operation is a drying cycle.

4. The operating method of the laundry device of claim 1, wherein the second step comprises steps of
identifying an operation object performing a refreshing cycle, and
opening a first steam supplying pipe, or a second steam supplying pipe connecting the identified operation object with the steam generating part.

5. The operating method of the laundry device of claim 4, wherein only the first steam supplying pipe connected with the washing machine with a drying finction is opened, once the operation object is identified as the washing machine with a drying function.

6. The operating method of the laundry device of claim 4, wherein only the second steam supplying pipe connected with the cabinet dryer is opened, once the operation object is identified as the cabinet dryer.

7. The operating method of the laundry device of claim 4, wherein the first and second steam supplying pipes are opened, once the operation object is identified as the washing machine with a drying function and the cabinet dryer.

8. The operating method of the laundry device of claim 7, wherein an opening range of each first and second steam supplying pipe is as various as necessary.

9. An operating method of a laundry device comprising:
a first step of a washing cycle for washing the laundry by using a washing machine with a drying function, out of the washing machine with a drying function and the cabinet dryer using the same drying heater, fan and steam generating part together;
a second step of a drying cycle for drying the laundry by using the washing machine with a drying function or the cabinet dryer; and
a third step of a refreshing cycle for refreshing the laundry by supplying steam into at least one of the washing machine with a drying function and the cabinet dryer, after drying the laundry.

10. The operating method of the laundry device of claim 9, wherein the first step comprises a step of drying another laundry within the cabinet dryer by using the cabinet dryer.

11. The operating method of the laundry device of claim 10, wherein the step of drying another laundry within the cabinet dryer by using the cabinet dryer is performed together with the first step of a washing cycle by using the washing machine with a drying function.

12. The operating method of the laundry device of claim 9, wherein the first step comprises a step of a refreshing cycle for another laundry within the cabinet dryer by using the cabinet dryer.

13. The operating method of the laundry device of claim 12, wherein the step of a refreshing cycle for another laundry within the cabinet dryer by using the cabinet dryer is performed together with the first step of a washing cycle by using the washing machine with a drying function.

14. The operating method of the laundry device of claim 9, wherein the second step comprises a step of a washing, a drying or a refreshing cycle by using one of the washing machine with a drying function or the cabinet dryer, which is not performing a drying cycle.

15. The operating method of the laundry device of claim 14, wherein the cycle by using one of the washing machine with a drying function and the cabinet dryer is performed with a drying cycle by using the other one at the same time.

16. The operating method of the laundry device of claim 9, wherein the third step comprises a step of a washing, drying or refreshing cycle by using one of the washing machine with a drying function and the cabinet dryer which is not performing a refreshing cycle.

17. The operating method of the laundry device of claim 16, wherein the cycle by using one of the washing machine with a drying function and the cabinet dryer is performed with a refreshing cycle by using the other one at the same time.
